(19) United States
(12) Patent Application Publication
(10) Pub. No.: US 2010/0009383 A1
(43) Pub. Date: Jan. 14, 2010

(54) Method for the simple and rapid detection of cells and biomolecules by means of paramagnetic particles

(76) Inventors: Holger Kiesewetter, Berlin (DE); Abdulgabar Salama, Berlin (DE); Jean Adam, Cressier s/Morat (CH)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/632,386

(22) PCT Filed: Jul. 12, 2005
(86) PCT No.: PCT/EP05/07571

§ 371 Date: Apr. 15, 2008

(30) Foreign Application Priority Data
Jul. 12, 2004 (DE) 10 2004 033 811.6

(51) Int. Cl. G01N 33/53; C12Q 1/02; G01N 33/553

(52) U.S. Cl. 435/7.2; 435/29; 436/526; 977/773; 977/920

(57) ABSTRACT

The invention relates to a simple and rapid method for the detection of cells and biomolecules by means of paramagnetic particles (beads) without separating the detected biomolecules or target cells from the beads before evaluating the specific bond.

The present invention relates to a simple and rapid method for the detection of cells and biomolecules by means of paramagnetic particles (beads). Paramagnetic beads (commercially available from various manufacturers) loaded with monospecific antibodies or antigens (detection molecules) are added to the sample to be investigated and mixed therewith. The sample with the beads is then put into a strong magnetic field. The magnetic field arrests the beads, and the remainder of the sample is completely removed or aspirated. The particles are suspended following removal of the magnetic field. Lumps formed by said particles then indicate the presence of a specific reaction or specific isolation of target cells or target biomolecules, since the latter react with the specific detection molecules and, together with the beads, form a visible linking (agglutination). The presence of a specific reaction may furthermore also be detected by photometrically determining binding of the specific biomolecule. In this case, use is made preferably of labeled beads or antibodies.

The method is characterized by extremely high sensitivity, simple implementation, rapid assaying and evaluation at any time and any place. The reaction may be enhanced visibly or massively by adding other, non-paramagnetic and smaller beads which are also loaded with specific detection molecules. Further detection or enhancement of the reaction is possible by assaying in the gel card system (particle agglutination test system) or by photometric measurement, in particular in the case of colored or europium-labeled beads.

Detection of cells and biomolecules is necessary in numerous fields, for example detection of infant cells in maternal circulation, early detection of tumor cells in the circulation of affected patients, detection of tumor cells in autologous stem cell preparations, detection of proteins (molecules in body fluid or tissue), detection of bacteria or germs in samples or food, detection of toxins (biological weapons) etc. The sensitive techniques used to date are relatively complicated and can be carried out only in special laboratories, such as for example flow cytometry (FACS), polymerase chain reaction (PCR) and immunohistochemistry [1-16]. Paramagnetic and non-paramagnetic micro- and nanobeads are supplied by various manufacturers (Dynal, Miltenyi, Biotec and others), and methods of purifying cells and biomolecules with the aid of paramagnetic beads have been disclosed [DE 101 11 520 A1; WO 02/090565 A2; DE 101 37 665 A1; US 2002/0072129 A1; EP 0 855 441 A2; US 2003/0175691 A1; U.S. Pat. No. 4,649,116; US 2003/0032028 A1; US 2004/0005718 A1]. Paramagnetic beads of this kind can be used for isolating from a solution and characterizing particular cells or molecules.

In a typical protocol, a suspension of magnetic particles is admixed with a sample containing particular cells or molecules. Subsequently, a magnetic field is applied so that the particles with the target cells or target molecules are arrested by the magnetic field on a wall of the vessel. The supernatant is discarded, and the particles are washed at least once more. A suitable buffer for separating the isolated cells or biomolecules from the beads is then added. Separation is carried out by centrifugation or by applying a magnetic field. The separated target molecules are detected by the abovementioned, relatively complicated methods such as, for example, FACS, PCR or immunohistochemical methods. In this connection, all of the methods described thus far have the following disadvantages:
1. the always required removal of the target molecules from the beads,
2. the loss of target molecules due to said removal,
3. the amount of work,
4. the amount of time,
4. the costs,
6. the usually complicated workability, and
7. the requirement of special equipment.

It was therefore the object of the present invention to develop a method which can be used to overcome the disadvantages of the above-discussed prior art. More specifically, the method should be rapid and simple to implement and, at the same time, be highly sensitive.

In a first aspect, the invention therefore relates to a method for detecting cells or biomolecules in a sample, which comprises:
(a) coating paramagnetic beads with a detection molecule (preferably a specific antibody or an antigen) to a feature of the biomolecule to be detected or the cell to be detected,
(b) contacting the sample to be investigated with the coated beads,
(c) removing the sample from the now loaded beads,
(d) evaluating the presence of the biomolecule to be detected or the cell to be detected on the basis of the presence of a specific reaction.

This method is advantageously carried out in such a way that in stage a) and/or b), in addition to the microbeads, nanobeads or antibodies coated with a second detection molecule which is directed either to the same feature or to another feature on the search cell/the biomolecule are carried as “agglutination enhancer” (or enhancer beads) in the binding reaction.

Alternatively, in a further preferred embodiment of the present invention, such agglutination enhancers may also be added only after stage (c) of the method of the invention.

The assay may preferably be carried out in the gel card system (particle agglutination test system).

According to the invention, nanoparticles are preferably from 50 nm to 0.5 μm, preferably about 100 nm, in size.

Particular preference is given to colored nanoparticles and/or microbeads, making a photometric evaluation possible.

A system for detection, which is particularly preferred according to the invention, comprises europium-containing beads, in particular nanobeads or else antibodies. Said beads and therefore the agglutinated particles formed can be detected in a highly sensitive manner by means of time-delayed fluorescence. Europium nanoparticles may be purchased, for example, from Seradyn, Indianapolis, USA. These particles are “soaked” with europium chelates so that the surface remains free for coupling reactions and the europium chelates do not leak out. Europium chelate: tris-(naphthyltrifluorobutanedione). These particles, when excited with UV light (maximum at 333 nm), emit light at 613 nm for approximately 0.5 milli-seconds, which is 10 000 times to 100 000 times longer than the emission of most fluorophores. This extremely long-lasting emission and the large Stokes' shift (difference between emission and excitation wavelengths) allows their use in assays based on time-delayed fluorescence. Each particle contains >30 000 europium atoms enclosed in tris-naphthyltrifluorobutanedione (a diketone). The “quantum yield” of the 100 nm particles is equivalent to approximately 3000 molecules of fluoresceine (one of the most commonly used fluorophores). In comparison, phycobiliprotein (probably the most fluorescent known compound) has a quantum yield corresponding to that of approx. 30 fluoresceine molecules. Since a 100 nm particle is 10 times larger in diameter than phycobiliprotein and has a 1000 times larger volume-to-mass ratio, the fluorescence of said beads is, on a molar basis, 100 times higher than that of phycobiliprotein.

Microparticles of the invention are between 0.1 to 5 μm, preferably 2.5 to 3 μm, in size.

The presence of an agglutination is advantageously evaluated visually or photometrically.

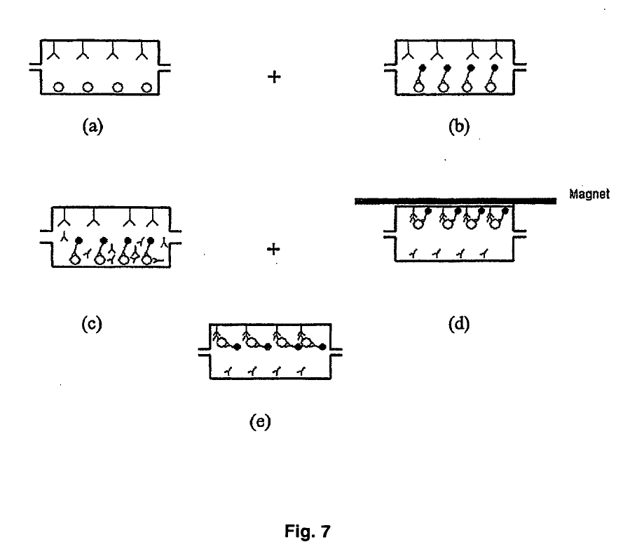

Optionally it is also possible to carry out one or more washing steps between stages (c) and (d). This (these) washing step(s) can advantageously be dispensed with, if the biomolecules to be detected are antibodies and a procedure according to FIG. 7 is chosen.

Biomolecules of the invention are in particular antigens and antibodies, but in principle it is possible to detect in this manner any biomolecules familiar to the skilled worker, such as proteins, nucleic acids and others. Said antibodies are in particular those which are detected, for example, in the determination of blood groups.

Detection molecules of the invention naturally depend on the specific biomolecule to be detected. Said detection molecules are preferably specific antibodies or antigens but may also be other binding molecules such as, for example, nucleic acids etc.

The expression “feature of the biomolecule to be detected or the cell to be detected (alternative name: target cell, abbreviation: ZZ)” means according to the invention, for example, an antigenic determinant (epitope) of said biomolecule or said cell to be detected. However, it may also be, for example, a complementary nucleic acid or other binding principles.

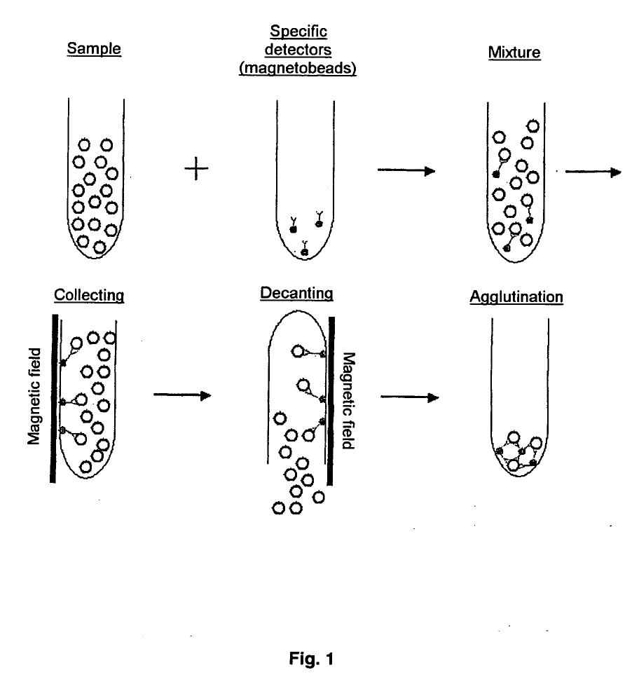

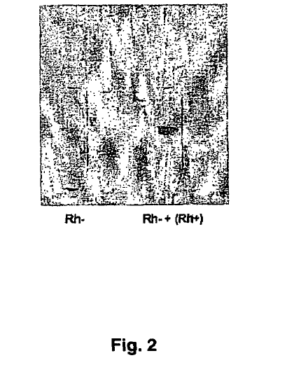

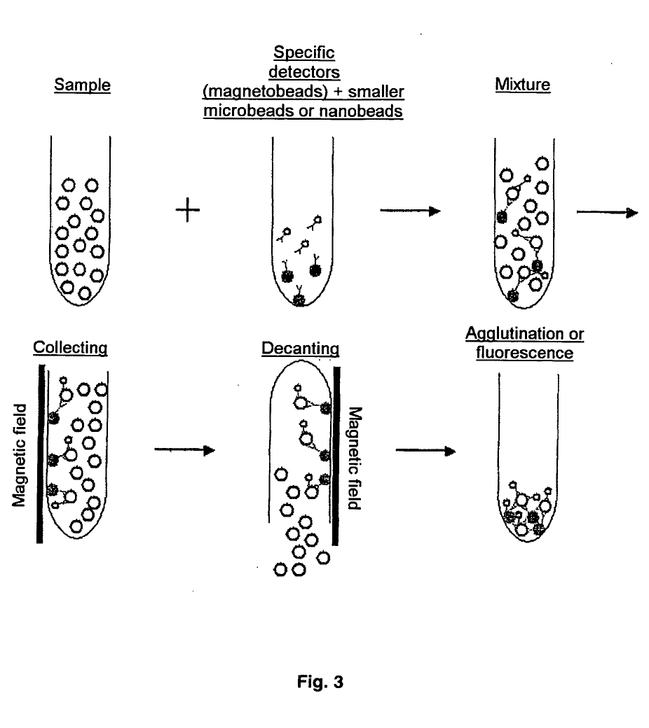

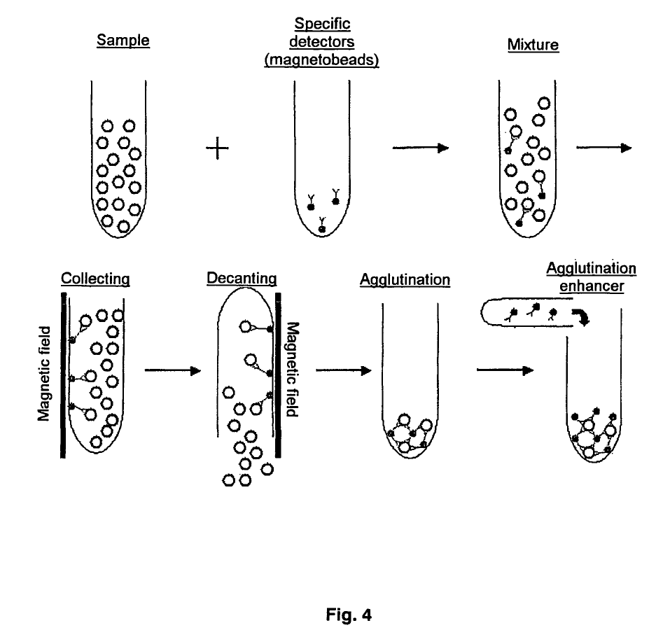

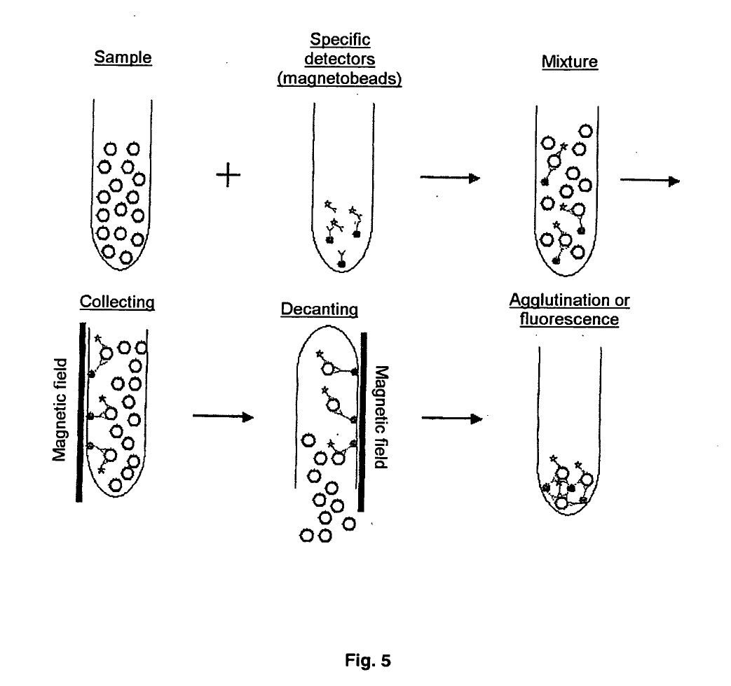

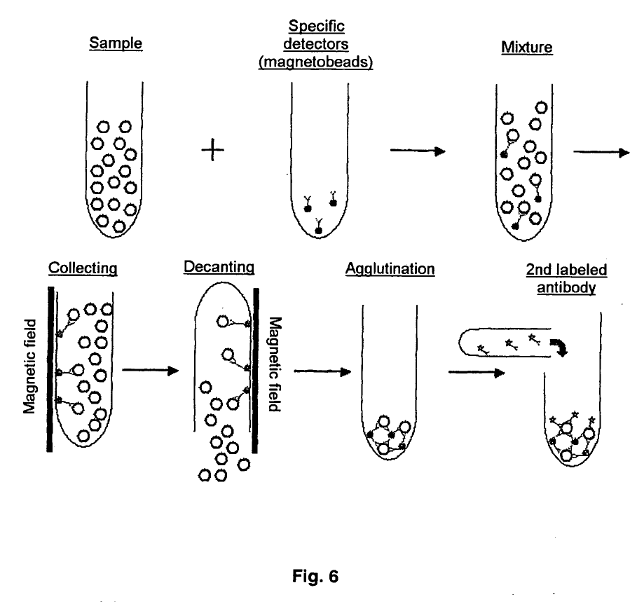

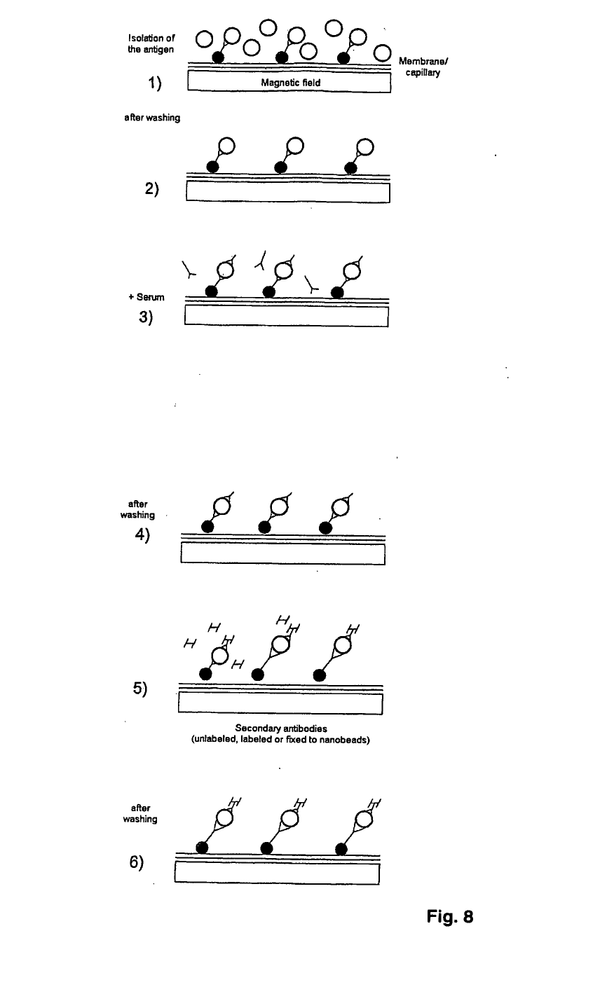

The method according to the invention overcomes the disadvantages of the prior art in that the isolated cells or biomolecules are not separated from the particles but that the reaction, preferably an agglutination, between the components is directly evaluated visually or photometrically (see FIG. 1). If an agglutination or a specific binding reaction which does not result in agglutination of the particles occurs, then this is a specific reaction and the sample is considered positive. The sensitivity may be increased by the amounts added and by using commercially available gel cards for detecting erythrocytic agglutination or particle agglutination (e.g. the ID card system from DiaMed) (FIG. 2). Visualization of the reactions may be further enhanced by adding smaller beads (nanobeads) (50 nm-0.5 μm) (FIG. 3). These enhancer beads are preloaded with antibodies or antigens to the same biomolecule or the same search cell. Depending on the method of measurement, colorless or colored enhancer beads may be used. In the latter case, the reactions may also be measured photometrically. The enhancer beads may be added to the starting sample either simultaneously (FIG. 3) with the paramagnetic beads or after isolation and purification (FIG. 4) of the target molecule. It is also possible to use labeled antibodies rather than enhancer beads, it being possible to use the labeling methods known in the prior art. A non-radioactive label is preferred here (FIG. 5 and FIG. 6). The reactions may be read visually, automatically or photometrically. In addition, the method is suitable for detecting not only antigenic biomolecules but also antibodies. Here, the washing process for removing free specific or unspecific unbound antibodies can be dispensed with (FIG. 7). But washing is also possible (FIG. 8).

In a preferred aspect, the present invention therefore relates to a method for detecting specific antibodies in a sample, which comprises, in addition to the abovementioned stage a), an additional stage a1) in which an antigen to which the antibody to be detected is directed is introduced on a support (I), without being tightly bound to said support, an additional stage a2) in which another support (II) is coated with an anti-species X antibody, species X being the species from which the antibody to be detected originates and the support being arranged in such a way that the sample to be investigated can contact in step b) initially only support (I), an additional stage a3) in which the coated beads of stage a) are contacted with the antigen of stage a1), and the magnet in stage c) is applied in such a way that, due to the magnetic force, the conglomerates of coated beads, antigen and antibody to be detected are moved toward the anti-X-coated support, with the presence of the antibody to be detected in the sample being determined by way of formation of specific agglutinated particles or specific binding reactions between said antibody to be detected and the anti-X antibody. This procedure is diagrammatically illustrated in FIG. 7. In this context, the covering plate may also be applied only prior to stage c). Binding is detected by means of the methods which are suitable and have been disclosed according to the invention.

Another, very important advantage of the method of the invention is the fact that the volume of the sample to be investigated can be increased virtually arbitrarily. Even sample volumes of up to 100 ml and more do not constitute a problem, owing to the immediate use of paramagnetic beads and, as a result, the possibility of dispensing with the separation of sample and detected molecule or ZZ. A theoretical upper limit of the sample size, which may be contemplated, is the availability of the magnetic beads used. For example, blood tests are carried out only in volumes of up to 100 μl in the prior art. This can be seen as a cause for the low sensitivity of the previous assays, which is decidedly improved by the present invention.

FIG. 1 depicts the procedure of the method of the invention.

FIG. 2 depicts the result of carrying out the method of the invention using an NaCl gel card from DiaMed, Switzerland.

FIG. 3 depicts the procedure of the method of the invention using agglutination enhancers introduced simultaneously with the paramagnetic beads.

FIG. 4 depicts the procedure of the method of the invention using agglutination enhancers added to the biomolecule or the cell after isolation and purification of the latter.

FIG. 5 depicts a possible procedure of the method of the invention using antibodies as agglutination enhancers.

FIG. 6 depicts another possible procedure of the method of the invention using antibodies as agglutination enhancers.

FIG. 7 depicts the procedure of the method of the invention for detecting antibodies. Antibodies (AB) to a particular target cell are to be detected. For this purpose, said particular target cell is introduced on a support material without binding, for example by pipetting into the well of a microtiter plate. Anti-human antibodies are tightly bound to the corresponding covering plate. Subsequently, paramagnetic beads coupled to a non-human AB to another ZZ-specific antigen are added and bind to the ZZ. This is followed by adding the human sample to be investigated, symbolized in FIG. 7 by the small drawn AB which, at the same time, represent any target cell-specific AB present in the sample, which, if present, are to be detected. If the sample contains AB to the ZZ, they will bind to said ZZ. Subsequently, the conglomerates formed in this way are “lifted” with the aid of the magnet so that said conglomerates contact the anti-human AB present on the bottom side of the cover. The conglomerates contain human AB only if the latter have bound as specific AB to the ZZ. Then, mediated via these AB and the anti-human AB, a specific binding reaction, in particular an agglutination, occurs which can be evaluated directly visually or else photometrically, depending on the labeling of the molecules and/or beads involved. A washing step is not required here.

FIG. 8 depicts the procedure of the method of the invention for detecting antibodies. Here, AB are likewise detected. To increase sensitivity and to avoid unspecific binding, a washing step is carried out here, however. The presence of specific AB in the serum is determined on the basis of the presence of an agglutination reaction.

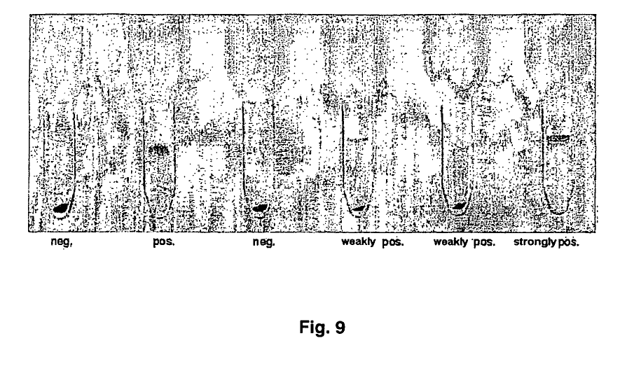

FIG. 9 depicts the result of example 1. The fact that anti-p53-containing sera result in agglutination of p53-coated beads is clearly visible.

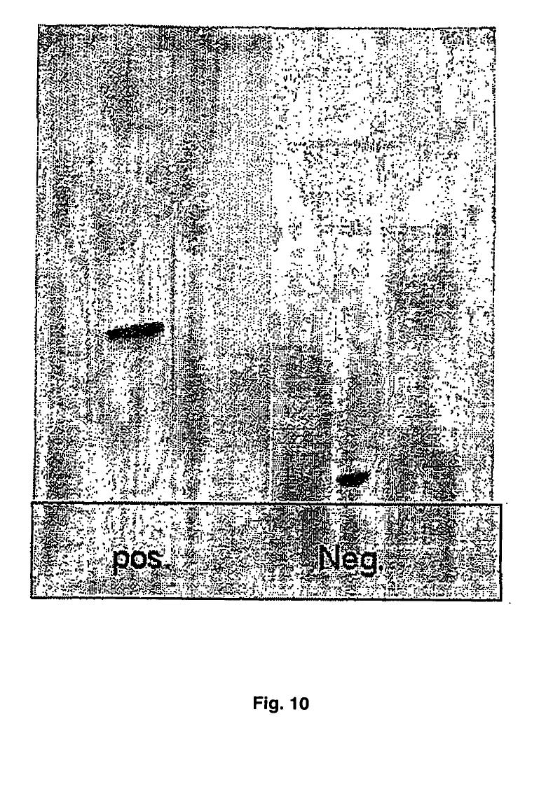

FIG. 10 depicts the result of example 2. The fact that IgG-containing sera result in agglutination of anti-IgG-coated beads is clearly visible.

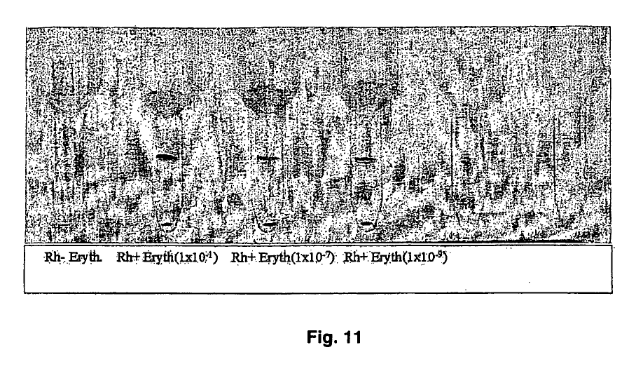

FIG. 11 depicts the result of example 3. The fact that Rhesus-positive (Rh⁺) erythrocytes react with anti-Rh⁺-coated beads and result in agglutination is clearly visible.

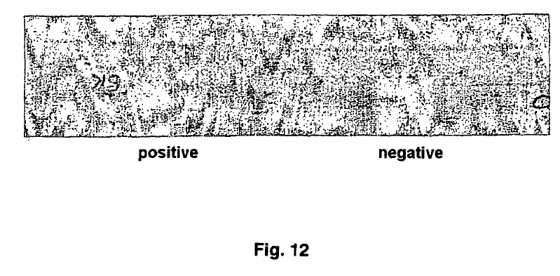

FIG. 12 depicts the result of example 4. The fact that anti-p53 AB result in agglutination of the beads in the tube is clearly visible.

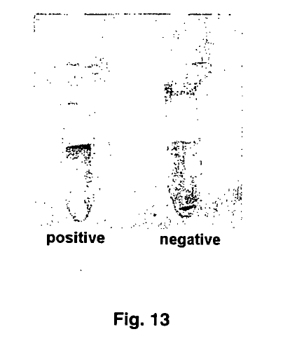

FIG. 13 depicts the result of example 5. The fact that autoantibodies to thrombocytes (platelets) result in agglutination of platelet antigen-coated beads is clearly visible.

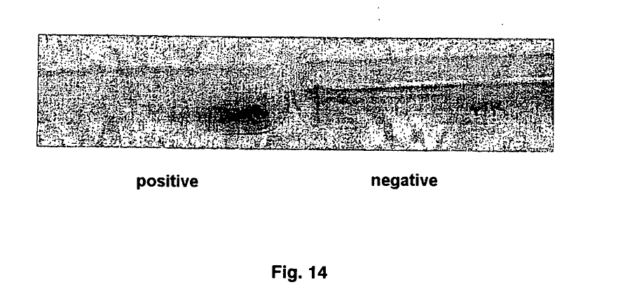

FIG. 14 depicts the result of example 6. The fact that Rh⁺ erythrocytes react with anti-RH⁺-coated beads and result in agglutination is clearly visible.

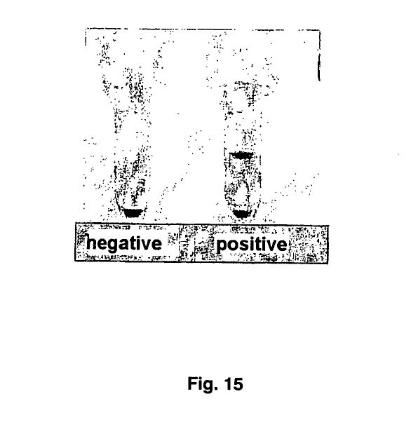

FIG. 15 depicts the result of example 7. The fact that the AB results in agglutination in the gel card (AB to T47D) is clearly visible.

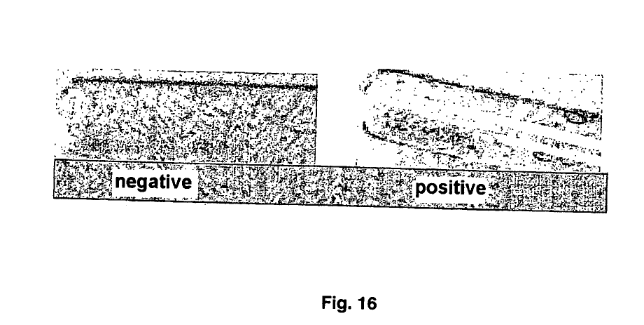

FIG. 16 depicts the result of example 7. The fact that AB results in agglutination in the tube (AB to T47D) is clearly visible.

The invention will be illustrated below on the basis of examples which, however, are not to be understood as being limiting.

### EXAMPLE 1

### Detection of Anti-P53

Beads (Dynal, Germany, Magnetobeads M-270, 2.8 μm in diameter) were coupled to human P53 (lys320-acetylated) (Biotrend, Cologne, Germany) according to the manufacturer's information.
40 μl of 0.1% beads (v/v) were incubated with 20 μl of serum (both from cancer patients and from healthy patients as control) at 37° C. for 30 min.
The beads were washed with 1× particle buffer (DiaMed, Switzerland) and resuspended in 50 μl of particle buffer.
The beads were applied in a layer on a gel card (DiaMed, “goat-anti-human”) 1:2 according to the manufacturer's information and centrifuged.

The sera used, 1, 2, 3 and 4, were from patients suspected of having cancer.

The result is depicted in FIG. 9. Anti-P53 AB was detected in the serum of patients without problems.

### EXAMPLE 2

### Detection of IgG

The beads (see example 1) were coupled to anti-human IgG (Dianova).
An IgG-containing sample (patient's serum) was incubated with 50 μl of beads (0.1% v/v) on an NaCl gel card (DiaMed, Switzerland) at 37° C. for 15 min and then centrifuged (FIG. 10).

Result: FIG. 10 clearly reveals that IgG-containing sera result in agglutination of anti-IgG-coated beads.

### EXAMPLE 3

### Detection of Rh⁺ cells in a Rh-negative (Rh⁻) Blood Sample (Total Amount: 3 ml)

The sample was mixed simultaneously with magnetobeads (see example 1) and nanobeads (Mikropartikel, Berlin, diameter 300 nm) at room temperature for approx. 5 min. Both types of beads are loaded with anti-D antibody (clone BRAD3, Bristol, England). For this purpose, 20 μg of AB were mixed with 100 μl of beads (10⁹ beads) and treated according to the manufacturer's information. The further procedure was as in examples 1 and 2.

Result: The sensitivity is incredibly high. One Rh⁺ erythrocyte can be detected among 10 000 000 000 other cells.

### EXAMPLE 4

### Detection of Anti-p53 in a Coumbs Tube

1) Dynalbeads (see example 1) were coated with human P53 and incubated with serum at 37° for 30 min.
2) The beads were washed 1× with particle buffer.
3) The beads were resuspended in 100 μl of particle buffer.
4) To this, 10 μl of nanobeads (100 nm in diameter, rabbit anti-human) were added and the mixture was centrifuged in a Coumbs centrifuge (manufacturer: Immuncore) at 300 g for 30 s (FIG. 12).

Result: The fact that anti-p53 AB result in agglutination of the beads in the tube is clearly visible.

### EXAMPLE 5

### Detection of Thrombocytic Antibodies with the Aid of a Gel Card

1) Dynalbeads (see example 1) were coated with AB anti-CD41 or anti-CD49b (monoclonal antibodies to thrombocytic antigens) and incubated with thrombocyte solubilizate (40 μl of concentrate) and 20 μl of serum at 37° C. for 30 min.
2) The beads were washed 1× with particle buffer.
3) The beads were resuspended in 50 μl of particle buffer and applied to a goat-anti-human gel card (Dynal) 1:4, according to the manufacturer's information (FIG. 13). The further procedure was as in examples 1 and 2.

Result: The fact that autoantibodies to thrombocytes (platelets) result in agglutination of the beads coated with platelet antigens is clearly visible.

### EXAMPLE 6

### Detection of RH⁺ Cells in a Coumbs Tube

1) 100 μl of Dynalbeads (see example 1) were coated with anti-D (example 3) and incubated with 10 μl anti-D-coated nanobeads (see example 4, 100 μm in diameter) for 10 min.
2) The beads were isolated from the blood using the magnet.
3) The beads were washed 1× with NaCl.
4) The beads were resuspended in 100 μl of NaCl and centrifuged in a Coumbs centrifuge (Immuncore) at 300 g for 30 s (FIG. 14).

Result: The fact that Rh⁺ erythrocytes react with anti-Rh⁺ (anti=D)-coated beads and result in agglutination is clearly visible.

### EXAMPLE 7

### Detection of Epithelial Tumor Cells (Cell Line T47D) in 2 ml of EDTA Blood by Clone 5E11, Both with the Aid of a Gel Card (DiaMed) and Directly in Blood

1) 10 μl of the antibody which had been diluted 1:5 and biotinylated according to standard methods (clone 5E11, Dianova EP4), were coupled to 10 μl of streptavidin beads (Dynal M-280) in an incubator at 37° C. for 0.5 h.
2) The beads were washed 2× with 100 μl of particle buffer.
3) The beads are resuspended in 100 μl of particle buffer and added to 2 ml of EDTA blood containing T47D cells (as positive sample) or to 2 ml of EDTA blood without T47D (negative sample).
4) 15 min of incubation on the rotor.
5) Then separation on the magnet for 10 min.
6) The beads were resuspended in 50 μl of particle buffer (agglutinated particles formed in the positive sample in this step) and (a) applied to a card (Dynal) (FIG. 15) and centrifuged in the card centrifuge for 10 min and (b) detected in the tube (FIG. 16).

Result: The fact that the AB results in agglutination in the gel card (AB to T47D, FIG. 15) is clearly visible. It is also clearly visible that the AB results in agglutination in the tube (AB to T47D9, FIG. 16).

1-16. (canceled)

17. A method for detecting cells or biomolecules in a sample, comprising:
a) coating paramagnetic microbeads with a detection molecule directed to a feature of said biomolecules or said cells,
b) contacting the sample with the coated microbeads, thereby loading the microbeads,
c) removing the loaded microbeads from the sample with the aid of a magnet,
d) resuspending the loaded microbeads removed from the sample in a buffer or a salt solution, the resuspension volume being smaller than the original sample volume,
e) applying the microbeads suspension obtained in step d) to a gel card or Coombs tube, thereby loading the gel card or the Coombs tube,
f) centrifuging the gel card or the Coombs tube loaded with the microbeads suspension, and
g) determining, visually or photometrically, the presence of the biomolecule or the cell on the basis of the presence of an agglutination between the biomolecules and/or the cell and the coated microbeads.

18. The method of claim 17, wherein the microbeads are between 0.1 and 5 μm.

19. The method of claim 18 wherein the microbeads are between 2.5 and 3 μm, in size.

20. The method of claim 18 wherein the microbeads are colored or labeled with europium.

21. The method of claim 17, wherein the buffer or salt solution further comprises nanobeads.

22. The method of claim 21, wherein the nanobeads are from 50 nm to 0.5 μm.

23. The method of claim 22 wherein the nanobeads are about 100 nm in size.

24. The method of claim 21 wherein the nanobeads are colored or europium labeled.

25. The method of claim 17, wherein a Coombs tube is utilized in step e) through step g).

26. The method of claim 17, wherein a gel card is utilized in step e) through step g).

27. A method for detecting cells or biomolecules in a sample, comprising:
a) loading paramagnetic microbeads (support I) with a specific antibody to a feature of said cells or said biomolecules,
b) introducing into the sample an antigen cognate to the cells or biomolecules to be detected;
c) loading a second support with an anti-X antibody, X being the species from which the cells or biomolecules to be detected originates, and species X differing from the species from which the antibody loaded on the microbeads originates,
d) contacting the loaded microbeads produced in step a) with the antigen of step b), while not allowing contact between the antigen and the second support;
e) removing the loaded microbeads from the sample with the aid of a magnet, using the magnetic force to move the conglomerates of loaded beads, and the cells or biomolecules to be detected toward the anti-X-loaded support II,
f) detecting the presence of the cells or biomolecules to be detected in the sample by way of formation of a specific bond between the antibody loaded on support I and the anti-X antibody loaded on support II.
